(19) United States
(12) Patent Application Publication
(10) Pub. No.: US 2010/0260755 A1
(43) Pub. Date: Oct. 14, 2010

(54) IBUDILAST AND IMMUNOMODULATORS COMBINATION

(75) Inventors: Richard E. GAMMANS, Vass, NC (US); Kenneth W. Locke, Carlsbad, CA (US); Michael E. Kalafer, San Diego, CA (US)

Correspondence Address: FOLEY AND LARDNER LLP;SUITE 500, 3000 K STREET NW, WASHINGTON, DC, 20007, US

(73) Assignee: MediciNova, Inc.

(21) Appl. No.: 12/755,939

(22) Filed: Apr. 7, 2010

Related U.S. Application Data
(60) Provisional application No. 61/168,108, filed on Apr. 9, 2009.

(51) Int. Cl. A61K 31/437; A61K 38/17; A61K 31/225; A61K 31/7076; A61K 31/4704; A61K 31/137; A61K 39/395; A61K 38/02; A61P 37/06; A61P 25/00

(52) U.S. Cl. 424/133.1; 514/17.9; 514/547; 514/46; 514/312; 514/653; 514/300

(57) ABSTRACT

The invention contemplates methods and compositions for treating multiple sclerosis including the administration of a PDE inhibitor and at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues, in a therapeutically effective amount. A preferred PDE inhibitor includes ibudilast.

### CROSS-REFERENCE TO RELATED APPLICATIONS

This application claims priority to U.S. Provisional Patent Application No. 61/168,108 filed Apr. 9, 2009, the entire contents of which are incorporated herein by reference.

### TECHNICAL FIELD

This invention relates to methods and compositions for treating multiple sclerosis comprising administering at least one PDE inhibitor, e.g., ibudilast, and at least one immunomodulator comprising mitoxantrone, natalizumab, fingolimod, laquinimod, cladribine, dimethylfumarate or a mixture comprising synthetic polypeptide analogs of myelin basic protein, thereof in a therapeutically effective amount.

### BACKGROUND

Multiple sclerosis (MS) is a disease in which the immune system attacks the central nervous system, leading to demyelination. It can be slowly progressive and has been characterized by diffuse patches of demyelination in the brain and spinal cord, resulting in multiple and varied neurologic symptoms and signs including changes in sensation; muscle weakness, muscle spasms, or difficulty in moving; difficulties with coordination and balance; problems in speech or swallowing, visual problems; fatigue, acute or chronic pain; and bladder and bowel difficulties. These varied neurologic symptoms and signs are usually associated with repeated relapse and remission.

Although the pathogenesis of MS still remains to be elucidated, tumor necrosis factor alpha (TNFα) and/or free radicals (e.g., NO and superoxide) may play a critical role in development of inflammatory demyelination. MS is also considered to be mediated by type I helper T cells (Th1), which secrete interferon γ (IFNγ), interleukin-2 (IL-2), and TNFα. In order to differentiate to Th1, naive helper T cells require signals from antigen presenting cells. One of the most critical signals for this differentiation is IL-12. Therefore, suppression of IL-12 production by antigen-presenting cells may interfere with differentiation of Th1, resulting in suppression of Th1-mediated autoimmune diseases.

It has been reported that phosphodiesterase (PDE) inhibitors (e.g., ibudilast) significantly suppressed the microglial IL-12 production. In addition, ibudilast also suppressed interferon-γ production by myelin oligodendrocyte glycoprotein (MOG)-specific T cells reactivated with MOG in the presence of microglia. Thus, PDE inhibitors may be used to suppress differentiation of T helper 1 (Th1) in the CNS. (Suzumura A, Multiple Sclerosis, 2003, Vol. 9, No. 6, 574-578). Other report showed that ibudilast suppressed the production of nitric oxide (NO), reactive oxygen species, interleukin (IL)-1β, IL-6, and tumor necrosis factor (TNF)-α and enhanced the production of the inhibitory cytokine, IL-10, and additional neurotrophic factors, including nerve growth factor (NGF), glia-derived neurotrophic factor (GDNF), and neurotrophin (NT)-4 in activated microglia. Tetsuya, et al., Neuropharmacology 2004, 46, 3, 404-41. Besides ibudilast, which is used to treat the relapsing-remitting form of MS, other PDE inhibitors such as pentoxifylline and rolipram, are also reported to be possibly effective to treat MS (See, Rott et al., Eur. J. Immunol., 1993, 23, 1745; Nataf et al., Acta Neurol. Scand., 1993, 88, 97; Genain et al., Proc. Natl. Acad. Sci., 1995, 92, 3601; Sommer et al., Nature Med., 1995, 1, 244; Jung et al., J. Neuroimmunol., 1996, 68, 1; and Okuda et al., Immunopharmacol., 1996, 35, 141).

Currently, there are several disease-modifying therapies developed for the treatment of multiple sclerosis such as interferons including interferon b-1a (Avonex®) and interferon β-1b (Betaseron®), glatiramer acetate (Copaxone®), mitoxantrone (NOVANTRONE®), and natalizumab (Tysabri®).

Glatiramer acetate (also called copolymer-1), a mixture comprising synthetic polypeptide analogs of myelin basic protein (MBP), has been suggested as a potential therapeutic agent for multiple sclerosis since the early '70s (Eur. J. Immunol. 1971, 1:242; and J. Neurol. Sci. 1977, 31:433). For example, it was shown to suppress experimental autoimmune encephalomyelitis, or experimental allergic encephalomyelitis (EAE). Eur. J. Immunol. 1971, 1:242; U.S. Pat. No. 3,849,550. EAE is an animal model of brain inflammation, an inflammatory demyelinating disease of the central nervous system (CNS). Later, copolymer-1 was reported to be beneficial for human patients with the exacerbating-remitting form of multiple sclerosis (N. Engl. J. Med. 1987, 317:408). Patients treated with daily injections of copolymer-1 had fewer exacerbations and smaller increases in their disability status than the control patients. It has also been shown that glatiramer acetate can effectively suppress the production of inflammatory mediators such as TNF-α, nitric oxide, and superoxide by glial cells. (Kayhan, et al., Immunology letters 2003, 88, 185-192).

Interferon (IFN), a class of glycoproteins known as cytokine, is a natural proteins produced by the cells of the immune system of most vertebrates in response to challenges viruses, parasites or tumor cells. Interferons have been shown to have about a 18-38% reduction in the rate of MS relapses, and to slow the progression of disability in MS patients. The subtype Interferon beta-1a is produced by mammalian cells while Interferon beta-1b is produced in modified E. coli. It is believed that Interferon beta based drugs achieve their beneficial effect on MS progression via their anti-inflammatory properties. Studies have also shown that Interferon beta improves the integrity of the blood-brain barrier (BBB), which generally breaks down in MS patients, allowing increasing amounts of undesirable substances to reach the brain.

Mitoxantrone is an anthracenedione antineoplastic agent. Mitoxantrone effective slows the progression of secondary progressive MS and extends the time between relapses in relapsing-remitting MS and progressive relapsing MS. Mitoxantrone is administered to patient intravenously.

Natalizumab is a humanized monoclonal antibody against alpha-4 (α4) integrin, which is required for white blood cells to move into organs. Natalizumab's mechanism of action is believed to be the inhibition of immune cells from crossing blood vessel walls to reach affected organs. Natalizumab has proven effective in treating the symptoms of both multiple sclerosis and Crohn's disease, preventing relapse, vision loss, cognitive decline and improving quality of life in people with multiple sclerosis. It increases rates of remission and preventing relapse in Crohn's disease. Natalizumab is administered by intravenous infusion every 4 weeks.

Other immunomodulators that may be used for the treatment of MS include fingolimod (FTY720), laquinimod, cladribine and dimethylfumarate (BG-12). Fingolimod is a structural analogue of sphingosine and gets phosphorylated by sphingosine kinases in the cell. It is known as a sphingosine-1-phosphate receptor 1 modulator. Laquinimod, a 1,2-dihydroquinoline derivative, is a once-daily, orally administered immunomodulatory compound that is being developed as a disease-modifying treatment for MS. Cladribine (2-chlorodeoxyadenosine), a purine analog, is a synthetic anti-cancer agent that also suppresses the immune system. An oral pill form has been successfully tested for multiple sclerosis. Dimethylfumarate (BG-12), an α,β-unsaturated ester, reacts rapidly with the detoxifying agent glutathione by Michael addition. When administered orally, BG-12 does not survive long enough to be absorbed into blood. It is reported to have potential neuroprotective and anti-inflammatory effects according to Biogen Idec who conducted a phase IIb clinical trial for the treatment of relapsing-remitting multiple sclerosis. In clinical trials doses up to 240 mg tds of BG-12 have been effective in relapsing-remitting multiple sclerosis.

It has been unexpectedly discovered that close to maximal therapeutic effect for the treatment of MS can be achieved using a combination of PDE inhibitors, e.g., ibudilast and other immunomodulators such as mitoxantrone, natalizumab, fingolimod (FTY720), laquinimod, cladribine, dimethylfumarate (BG-12) or a mixture comprising synthetic polypeptide analogs of myelin basic protein, e.g., glatiramer acetate, as discussed further below.

### SUMMARY

According to one aspect of the present invention, methods are provided for treating a patient suffering from the negative effects of multiple sclerosis. The methods comprise administering a therapeutically effective amount of at least one phosphodiesterase inhibitor, its pharmacologically acceptable salt, or a hydrate or solvate of one of the foregoing (collectively, “PDE inhibitor”), and a therapeutically effective amount of at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues.

According to another aspect of the present invention, there is provided pharmaceutical compositions for treating a patient suffering from the negative effects of multiple sclerosis comprising: at least one phosphodiesterase inhibitor, its pharmacologically acceptable salt, or a hydrate or solvate of one of the foregoing (collectively, “PDE inhibitor”), and at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues.

According to yet another aspect of the present invention, there is provided methods of modulating effects of a mixture comprising synthetic polypeptide analogs of myelin basic protein on microglial production of an inflammatory mediator in a patient, comprising: co-administering at least one PDE inhibitor and a mixture comprising synthetic polypeptide analogs of myelin basic protein in an amount sufficient to reduce an increase in microglial production of an inflammatory mediator induced by the mixture comprising synthetic polypeptide analogs of myelin basic protein.

### DETAILED DESCRIPTION OF PREFERRED EMBODIMENTS

Various embodiments of the invention are described hereinafter. It should be noted that the specific embodiments are not intended as an exhaustive description of the invention or as a limitation on the scope of the invention. One aspect described in conjunction with a particular embodiment of the present invention is not necessarily limited to that embodiment and can be practiced with any other embodiment(s) of the invention.

In one aspect, the present invention provides methods of treating a patient suffering from the negative effects of multiple sclerosis. The methods comprise administering a therapeutically effective amount of at least one phosphodiesterase inhibitor, its pharmacologically acceptable salt, or a hydrate or solvate of one of the foregoing (collectively, “PDE inhibitor”), and a therapeutically effective amount of at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues. The administering step may comprise simultaneously or in close temporal proximity administering the at least one PDE inhibitor and the at least one immunomodulator. The at least one PDE inhibitor preferably comprises ibudilast. In one aspect, the at least one immunomodulator may comprise mitoxantrone. In another aspect, the at least one immunomodulator may comprise natalizumab. In yet another aspect, the at least one immunomodulator may comprise fingolimod. In yet another aspect, the at least one immunomodulator may comprise laquinimod. In yet another aspect, the at least one immunomodulator may comprise cladribine. In yet another aspect, the at least one immunomodulator may comprise dimethylfumarate.

In yet another aspect, the at least one immunomodulator may comprise synthetic polypeptide analogs of myelin basic protein, including alanine, glutamic acid, lysine, and tyrosine amino acid residues. In one aspect, the mixture may comprise synthetic polypeptide analogs of myelin basic protein, which are non-uniform with respect to molecular weight and amino acid sequence. In another aspect, the mixture comprises synthetic polypeptide analogs of myelin basic protein, which have an average molecular weight falling in the range of about 4 to about 9 kilodaltons (kDa). In yet another aspect, over 75% of the molar fraction of the mixture comprises synthetic polypeptide analogs of myelin basic protein having a molecular weight falling in the range of about 2 kDa to about 20 kDa. In yet another aspect, less than 5% of the molar fraction of the mixture comprises synthetic polypeptide analogs of myelin basic protein having a molecular weight exceeding 40 kDa. In yet another aspect, the mixture comprises synthetic polypeptide analogs of myelin basic protein, which have an average molecular weight falling in the range of about 6.25 to about 8.4 kDa. In yet another aspect, the mixture of synthetic polypeptide analogs of myelin basic protein has an average molecular weight of 5 to 9 kilodaltons. In yet another aspect, the mixture of synthetic polypeptide analogs of myelin basic protein comprises glatiramer acetate.

As used herein, the terms “therapeutic” and/or “effective” amounts mean an agent utilized in an amount sufficient to treat, combat, ameliorate, prevent or improve a condition or disease of a subject. A therapeutically effective amount can be readily determined by the attending diagnostician, as one skilled in the art, by the use of known techniques and by observing results obtained under analogous circumstances. In determining the therapeutically effective amount or dose, a number of factors are considered by the attending diagnostician, including, but not limited to: the species of mammal; its size, age, and general health; the specific disease involved; the degree of or involvement or the severity of the disease; the response of the individual subject; the particular compound administered; the mode of administration; the bioavailability characteristics of the preparation administered; the dose regimen selected; the use of concomitant medication; and other relevant circumstances. “Subject” refers to mammals and includes humans and non-human mammals. These disease conditions include MS.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

The mixture comprising synthetic polypeptide analogs of myelin basic protein, including alanine, glutamic acid, lysine, and tyrosine amino acid residues, according to the present invention, may be prepared by methods known in the art (e.g., U.S. Pat. No. 3,849,550). Such methods include chromatography of the mixture containing high molecular weight species and collecting the fractions without the undesired species or by partial acid or enzymatic hydrolysis to remove the high molecular weight species with subsequent purification by dialysis or ultrafiltration. A further method to obtain a mixture comprising synthetic polypeptide analogs of myelin basic protein with the desired molecular weight profile is by preparing the desired species while the amino acids are still protected (Teitelbaum et al., Eur. J. Immun. 1971, 1, 242) and then obtain the correct species directly upon removing the protection. The compositions of the mixture may be formulated by conventional methods known in the art. Preferably, the composition is lyophilized and formed into an aqueous solution suitable for sub-cutaneous injection. Alternatively, the mixture comprising synthetic polypeptide analogs of myelin basic protein may be formulated in any of the forms known in the art for preparing oral, nasal, buccal, or rectal formulations of peptide drugs.

In another aspect, the at least one pharmacologically or pharmaceutically acceptable salt in the methods of treating a patient suffering from the negative effects of multiple sclerosis may comprise an inorganic acid addition salt, an organic carboxylic acid addition salt, or a combination thereof. The inorganic acid addition salt is selected from one or more mineral acid addition salts. In one aspect, the one or more mineral acid addition salts may be selected from a hydrochloric acid addition salt, a sulfuric acid addition salt and a nitric acid addition salt. In another aspect, the organic carboxylic acid addition salt is selected from one or more of an acetic acid addition salt, a propionic acid addition salt, a maleic acid addition salt, a fumaric acid addition salt, an oxalic acid addition salt, a carboxysuccinic acid addition salt and a citric acid addition salt.

In yet another aspect, the methods of treating a patient suffering from the negative effects of MS comprising the at least one PDE inhibitor and the at least one immunomodulator comprising Mitoxantrone, Natalizumab, Fingolimod, Laquinimod, Cladribine, Dimethylfumarate or a mixture comprising synthetic polypeptide analogs of myelin basic protein, including alanine, glutamic acid, lysine, and tyrosine amino acid residues, may be in a form of a solution or a suspension. Preferably, the solution or suspension further comprises at least one of a sterile diluent, an antibacterial agent, an antioxidant, a chelating agent, a buffer and a tonicity adjusting agent, or combinations thereof. More preferably, the at least one PDE inhibitor may comprise ibudilast. In another aspect, the at least one PDE inhibitor may comprise Cilomilast, (5-[3-[(1S,2S,4R)-Bicyclo[2.2.1]hept-2-yloxy]-4-methoxyphenyl]tetrahydro-2(1H)-pyrimidinone) (CP-80633), Drotaverine, Etazolate, Glaucine, 5-(3-(cyclopentyloxy)-4-methoxyphenyl)-3-(3-methylbenzyl)piperidin-2-one (HT-0712), 2-Amino-6-methyl-4-propyl-[1,2,4]triazolo[1,5-a]pyrimidin-5(4H)-one (ICI-63197), Irsogladine, Mesembrine, Pentoxifylline, Roflumilast, Rolipram, 4-(3-Butoxy-4-methoxyphenyl)methyl-2-imidazolidone (Ro20-1724), N-{2-[(2E)-2-(mesitylimino)-9,10-dimethoxy-4-oxo-6,7-dihydro-2H-pyrimido[6,1-a]-isoquinolin-3(4H)-yl]ethyl}urea (RPL-554 or 4-(3-Chlorophenyl)-1,7-diethylpyrido[2,3-d]pyrimidin-2(1H)-one (YM-976). The mixture of comprising synthetic polypeptide analogs of myelin basic protein may comprise glatiramer acetate.

For example, for purposes of parenteral therapeutic administration, ibudilast and glatiramer acetate may be incorporated into a solution or a suspension. The amount of active compound in such compositions is such that a suitable dosage will be obtained. The Solution or suspension may also include the following components: a sterile diluent, such as water for injection, saline solution, fixed oils, polyethylene glycols, glycerine, propylene glycol or other synthetic solvents: antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity or osmolarity, such as sodium chloride or dextrose. The solution or suspension may be administered to a subject parenterally. The parenteral preparation may be enclosed in ampoules, disposable syringes or multiple dose vials made of glass or plastic.

In yet another aspect, the administering step comprises orally administering the at least one PDE inhibitor and at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues. Preferably, the at least one PDE inhibitor and the at least one immunomodulator are administered in a form of a tablet or a capsule. The at least one PDE inhibitor may comprise ibudilast. The mixture of comprising synthetic polypeptide analogs of myelin basic protein may comprise glatiramer acetate.

For example, ibudilast and glatiramer acetate may be administered orally, with an inert diluent, typically an edible carrier. They may be enclosed in gelatin capsules or compressed into tablets. For the purpose of oral therapeutic administration, the compounds may be incorporated with excipients and used in the form of tablets, troches, capsules, elixirs, suspensions, syrups, waters, chewing gums, and the like. The amount of the compounds consisting of embodiments of the present invention will be such that a suitable dosage will be provided in the administered amount.

Tablets, pills, capsules, troches and the like may contain the following ingredients: a binder, such as micro-crystalline cellulose, gum tragacanth or gelatin; an excipient, such as starch or lactose; a disintegrating agent, such as alginic acid, Primogel, corn starch and the like; a lubricant, such as magnesium stearate or Sterotes; a glidant, such as colloidal silicon dioxide; a sweetening agent, such as sucrose, saccharin or aspartame; or flavoring agent, such as peppermint, methyl salicylate or orange flavoring. When the dosage unit form is a capsule it may contain, in addition to compounds comprising embodiments of the present invention, a liquid carrier, such as a fatty oil. Other dosage unit forms may contain other materials that modify the physical form of the dosage unit, for example, as coatings. The coating(s) can be formulated for immediate release, delayed/enteric release or sustained release of the second pharmaceutical active in accordance with methods well known in the art. For example, a coating for immediate release is commonly used as a moisture barrier, and for taste and odor masking Rapid breakdown of the coating in gastric media will lead to effective disintegration and dissolution. Thus, tablets or pills may be coated with sugar, shellac or other enteric coating agents. A syrup may contain, in addition to the active compounds, sucrose as a sweetening agent and preservatives, dyes, colorings and flavors. Materials used in preparing these compositions should be pharmaceutically pure and non-toxic in the amounts used.

The dosage form of the present invention may be either immediate or controlled release. For example, an immediate release form may comprise one or more pharmaceutically acceptable excipients including, but not limited to, one or more of microcrystalline cellulose, hydroxypropylcellulose, starch, lactose monohydrate, anhydrous lactose, talc, colloidal silicon dioxide, povidone, citric acid, poloxamer, sodium starch glycolate, stearic acid, and magnesium stearate.

Controlled release can be achieved in the formulations by several mechanisms known in the art. For example, pH sensitive polymer or co-polymer can be used which when applied around the drug matrix functions as an effective barrier to release of active at certain pH range. An alternative to a pH sensitive polymer or co-polymer is a polymer or co-polymer that is non-aqueous-soluble. The extent of resistance to release, for example, in the gastric environment can be controlled by coating with a blend of the non-aqueous-soluble and a aqueous soluble polymer. In this approach neither of the blended polymers or co-polymers are pH sensitive. One example of a pH sensitive co-polymer is the Eudragit® methacrylic co-polymers, including Eudragit® L 100, S 100 or L 100-55 solids, L 30 D-55 or FS 30D dispersions, or the L 12.5 or S 12.5 organic solutions. The polymers may be applied to a tablet either by spray coating (as a thin film) or by compression coating. Polymer(s) may be applied over the surface of the capsule or applied to microparticles of the drug, which may then be encapsulated such as in a capsule or gel.

A sustained release film coat may be used for the invention compositions including a water insoluble material such as a wax or a wax-like substance, fatty alcohols, shellac, zein, hydrogenated vegetable oils, water insoluble celluloses, polymers of acrylic and/or methacrylic acid, and any other slowly digestible or dispersible solids known in the art.

Other means known in the art such as a swellable hydrogel may be used to delay release (an osmotic pump system). The swellable hydrogel takes up moisture after administration. Swelling of the gel results in displacement of the drug from the system for absorption. The timing and rate of release of the drug depend on the gel used, and the rate at which moisture reaches the gel, which can be controlled by the size of the opening in the system through which fluid enters. See Drug Delivery Technologies online article Dong et al., “L-OROS® SOFTCAP™ for Controlled Release of Non-Aqueous Liquid Formulations.

The phrase “pharmaceutically acceptable carrier” is art-recognized, and includes, for example, pharmaceutically acceptable materials, compositions or vehicles, such as a liquid or solid filler, diluent, solvent or encapsulating material, involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

According to the present invention, there is provided compositions for treating a patient suffering from the negative effects of multiple sclerosis comprising: at least one phosphodiesterase inhibitor, its pharmacologically acceptable salt, or a hydrate or solvate of one of the foregoing (collectively, “PDE inhibitor”), and at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues, present in a pharmaceutically acceptable carrier. The at least one PDE inhibitor preferably comprises ibudilast. In one aspect, the at least one immunomodulator may comprise mitoxantrone. In another aspect, the at least one immunomodulator may comprise natalizumab. In yet another aspect, the at least one immunomodulator may comprise fingolimod. In yet another aspect, the at least one immunomodulator may comprise laquinimod. In yet another aspect, the at least one immunomodulator may comprise cladribine. In yet another aspect, the at least one immunomodulator may comprise dimethylfumarate.

In yet another aspect, the at least one immunomodulator may comprise a mixture comprising synthetic polypeptide analogs of myelin basic protein, including alanine, glutamic acid, lysine, and tyrosine amino acid residues. In one aspect, the mixture comprises synthetic polypeptide analogs of myelin basic protein, which are non-uniform with respect to molecular weight and amino acid sequence. In another aspect, the mixture comprises synthetic polypeptide analogs of myelin basic protein, which have an average molecular weight falling in the range of about 4 to about 9 kilodaltons (kDa). In yet another aspect, over 75% of the molar fraction of the mixture comprises synthetic polypeptide analogs of myelin basic protein having a molecular weight falling in the range of about 2 kDa to about 20 kDa. In yet another aspect, less than 5% of the molar fraction of the mixture comprises synthetic polypeptide analogs of myelin basic protein having a molecular weight exceeding 40 kDa. In yet another aspect, the mixture comprises synthetic polypeptide analogs of myelin basic protein which have an average molecular weight falling in the range of about 6.25 to about 8.4 kDa. In yet another aspect, the mixture comprises synthetic polypeptide analogs of myelin basic protein which have an average molecular weight falling in the range of about 5 to about 9 kDa. In yet another aspect, the mixture may comprise glatiramer acetate.

In yet another aspect, the present invention provides compositions for treating a patient suffering from the negative effects of multiple sclerosis comprising at least one PDE inhibitor, and at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues, in a form of solution or suspension. The solution or suspension may further comprise at least one of a sterile diluent, an antibacterial agent, an antioxidant, a chelating agent, a buffer, a tonicity adjusting agent, or combinations thereof.

In yet another aspect, the present invention provides compositions for treating a patient suffering from the negative effects of multiple sclerosis wherein the at least one PDE inhibitor is administered in a solution or suspension at a concentration ranging from about 1 μM/mL to about 300 μM/mL and the mixture comprising synthetic polypeptide analogs of myelin basic protein is administered in the same solution at a specific activity ranging from about 1 mg/mL to about 100 mg/mL; preferably the at least one PDE inhibitor is administered in a solution at a concentration ranging from about 1 μM/mL to about 100 μM/mL and the mixture comprising synthetic polypeptide analogs of myelin basic protein is administered in the same solution at a specific activity ranging from about 20 mg/mL to about 40 mg/mL. The at least one PDE inhibitor and the mixture comprising synthetic polypeptide analogs of myelin basic protein may be administered in a solution or suspension in which the foregoing active ingredients are present in a weight ratio ranging from about 1:5 to about 5:1.

In yet another aspect, the present invention provides compositions for treating a patient suffering from the negative effects of multiple sclerosis wherein the at least one PDE inhibitor is administered in a tablet or capsule at a dosage ranging from about 20 mg to about 120 mg and the mixture comprising synthetic polypeptide analogs of myelin basic protein is administered in the same tablet or capsule at a specific activity ranging from about 1 mg to about 100 mg; preferably the at least one PDE inhibitor is administered in a solution at a tablet or capsule ranging from about 20 mg to about 80 mg and the mixture comprising synthetic polypeptide analogs of myelin basic protein is administered in the same tablet or capsule at a specific activity ranging from about 20 mg to about 40 mg. The at least one PDE inhibitor and the mixture comprising synthetic polypeptide analogs of myelin basic protein may be administered in a tablet or capsule in which the foregoing active ingredients are present in a weight ratio ranging from about 1:5 to about 5:1.

The present invention also provides methods of modulating effects of a mixture comprising synthetic polypeptide analogs of myelin basic protein on microglial production of an inflammatory mediator in a patient, comprising: co-administering at least one PDE inhibitor and a mixture comprising synthetic polypeptide analogs of myelin basic protein in an amount sufficient to reduce an increase in microglial production of an inflammatory mediator induced by the mixture comprising synthetic polypeptide analogs of myelin basic protein. In one aspect, the inflammatory mediator in the methods comprises Interleukin-5 (IL-5). In another aspect, the inflammatory mediator in the methods may comprise Interleukin-13 (IL-13). In yet another aspect, the microglial production in the methods is stimulated by lipopolysaccharide.

It was reported that glatiramer acetate induces IL-5 and IL-13 cytokine secretion in T-cells isolated from peripheral blood of MS patients in vitro (Wiesemann, et al., Clin. Exp. Immunol. 2003, 133, 454). On the other hand, PDE inhibitors were reported to suppressed IL-5 generation as shown in animal model (Souness, et al., Biochem. Phar. 1999, 58, 991-999). It can be realized unexpectedly by the present invention that PDE inhibitors, e.g., ibudilast and the mixture comprising synthetic polypeptide analogs of myelin basic protein, e.g. glatiramer acetate, can function additively or synergistically to suppress IL-5 and/or IL-13 production by microglia.

In yet another aspect, the methods of modulating effects of a mixture comprising synthetic polypeptide analogs of myelin basic protein on microglial production of an inflammatory mediator in a subject comprise at least one PDE inhibitor, which is administered in a solution or suspension at a concentration ranging from about 1 μM/mL to about 300 μM/mL and the mixture comprising synthetic polypeptide analogs of myelin basic protein, which is administered in the same solution at a specific activity ranging from about 1 mg/mL to about 100 mgU/mL; preferably the at least one PDE inhibitor is administered in a solution at a concentration ranging from about 1 μM/mL to about 100 μM/mL and the mixture comprising synthetic polypeptide analogs of myelin basic protein is administered in the same solution or suspension at a specific activity ranging from about 20 mg/mL to about 40 mg/mL In a related aspect, the methods of modulating effects of a mixture comprising synthetic polypeptide analogs of myelin basic protein on microglial production of an inflammatory mediator in a subject comprise at least one PDE inhibitor, which is administered in a solution a tablet or capsule at a dosage ranging from about 20 mg to about 120 mg and the mixture comprising synthetic polypeptide analogs of myelin basic protein is administered in the same tablet or capsule at a specific activity ranging from about 1 mg to about 100 mg; preferably the at least one PDE inhibitor is administered in a solution at a tablet or capsule ranging from about 20 mg to about 80 mg and the mixture comprising synthetic polypeptide analogs of myelin basic protein is administered in the same tablet or capsule at a specific activity ranging from about 20 mg to about 40 mg. The at least one PDE inhibitor and the mixture comprising synthetic polypeptide analogs of myelin basic protein may be present in a weight ratio ranging from about 1:5 to about 5:1. In yet another aspect, the subject is suffering from the negative effects of MS. The at least one PDE inhibitor may comprise ibudilast. In another aspect, the at least one PDE inhibitor may comprise Cilomilast, (5-[3-[(1S,2S,4R)-Bicyclo[2.2.1]hept-2-yloxy]-4-methoxyphenyl]tetrahydro-2(1H)-pyrimidinone) (CP-80633), Drotaverine, Etazolate, Glaucine, 5-(3-(cyclopentyloxy)-4-methoxyphenyl)-3-(3-methylbenzyl)piperidin-2-one (HT-0712), 2-Amino-6-methyl-4-propyl-[1,2,4]triazolo[1,5-a]pyrimidin-5(4H)-one (ICI-63197), Irsogladine, Mesembrine, Pentoxifylline, Roflumilast, Rolipram, 4-(3-Butoxy-4-methoxyphenyl)methyl-2-imidazolidone (Ro20-1724), N-{2-[(2E)-2-(mesitylimino)-9,10-dimethoxy-4-oxo-6,7-dihydro-2H-pyrimido[6,1-a]-isoquinolin-3(4H)-yl]ethyl}urea (RPL-554 or 4-(3-Chlorophenyl)-1,7-diethylpyrido[2,3-d]pyrimidin-2(1H)-one (YM-976). The mixture of comprising synthetic polypeptide analogs of myelin basic protein may comprise glatiramer acetate.

Obviously, numerous modifications and variations of the present invention are possible in light of the above teachings. It is therefore to be understood that within the scope of the appended claims, the invention may be practiced otherwise than as specifically described herein.

### EXAMPLES

The following examples are provided to illustrate certain aspects of the present invention and to aid those of skill in the art in practicing the invention. These examples are not intended to limit the scope of the invention.

### Example 1

### A Double-Blind, Randomized Study Comparing the Combined Use of Ibudilast and Glatiramer Acetate (GA) to Either Agent Alone in Patients with Multiple Sclerosis

This is a double blind, randomized trial examining combination therapy versus single agent therapy with long term (e.g. at least 12 months) follow-up on the last patient randomized. All patients will remain on therapy until the last patient completes the study. All patients will then be transitioned, based on the findings, to open label of combination with continued follow-up or some recommendation about single agent therapy. The primary interest is in combination therapy. Therefore, a two-group combination versus single agent concept will be used—splitting the population into single agent and combination therapy equally. The single agent arm is divided into two groups, ibudilast and GA providing for 3 treatment arms: ibudilast and GA (50% of the patients), ibudilast and placebo (25% of the patients) and GA and placebo (25% of the patients). The general exclusion criteria is followed. Ibudilast is administered in a solution or suspension at a concentration ranging from about 1 μM/mL to about 300 μM/mL and GA is administered in the same solution at a specific activity ranging from about 1 mg/mL to about 100 mg/mL.

### Example 2

### A Double-Blind, Randomized Study Comparing the Combined Use of Ibudilast and Mitoxantrone to Either Agent Alone in Patients with Multiple Sclerosis

This is a double blind, randomized trial examining combination therapy versus single agent therapy with long term (e.g. at least 12 months) follow-up on the last patient randomized. All patients will remain on therapy until the last patient completes the study. All patients will then be transitioned, based on the findings, to open label of combination with continued follow-up or some recommendation about single agent therapy. The primary interest is in combination therapy. Therefore, a two-group combination versus single agent concept will be used—splitting the population into single agent and combination therapy equally. The single agent arm is divided into two groups, ibudilast and mitoxantrone providing for 3 treatment arms: ibudilast and mitoxantrone (50% of the patients), ibudilast and placebo (25% of the patients) and mitoxantrone and placebo (25% of the patients). The general exclusion criteria is followed.

### Example 3

### A Double-Blind, Randomized Study Comparing the Combined Use of Ibudilast and Natalizumab to Either Agent Alone in Patients with Multiple Sclerosis

This is a double blind, randomized trial examining combination therapy versus single agent therapy with long term (e.g. at least 12 months) follow-up on the last patient randomized. All patients will remain on therapy until the last patient completes the study. All patients will then be transitioned, based on the findings, to open label of combination with continued follow-up or some recommendation about single agent therapy. The primary interest is in combination therapy. Therefore, a two-group combination versus single agent concept will be used—splitting the population into single agent and combination therapy equally. The single agent arm is divided into two groups, ibudilast and natalizumab providing for 3 treatment arms: ibudilast and natalizumab (50% of the patients), ibudilast and placebo (25% of the patients) and natalizumab and placebo (25% of the patients). The general exclusion criteria is followed.

### Example 4

### A Double-Blind, Randomized Study Comparing the Combined Use of Ibudilast and Fingolimod to Either Agent Alone in Patients with Multiple Sclerosis

This is a double blind, randomized trial examining combination therapy versus single agent therapy with long term (e.g. at least 12 months) follow-up on the last patient randomized. All patients will remain on therapy until the last patient completes the study. All patients will then be transitioned, based on the findings, to open label of combination with continued follow-up or some recommendation about single agent therapy. The primary interest is in combination therapy. Therefore, a two-group combination versus single agent concept will be used—splitting the population into single agent and combination therapy equally. The single agent arm is divided into two groups, ibudilast and fingolimod providing for 3 treatment arms: ibudilast and fingolimod (50% of the patients), ibudilast and placebo (25% of the patients) and fingolimod and placebo (25% of the patients). The general exclusion criteria is followed.

### Example 5

### A Double-Blind, Randomized Study Comparing the Combined Use of Ibudilast and Laquinimod to Either Agent Alone in Patients with Multiple Sclerosis

This is a double blind, randomized trial examining combination therapy versus single agent therapy with long term (e.g. at least 12 months) follow-up on the last patient randomized. All patients will remain on therapy until the last patient completes the study. All patients will then be transitioned, based on the findings, to open label of combination with continued follow-up or some recommendation about single agent therapy. The primary interest is in combination therapy. Therefore, a two-group combination versus single agent concept will be used—splitting the population into single agent and combination therapy equally. The single agent arm is divided into two groups, ibudilast and laquinimod providing for 3 treatment arms: ibudilast and laquinimod (50% of the patients), ibudilast and placebo (25% of the patients) and laquinimod and placebo (25% of the patients). The general exclusion criteria is followed.

### Example 6

### A Double-Blind, Randomized Study Comparing the Combined Use of Ibudilast and Cladribine to Either Agent Alone in Patients with Multiple Sclerosis

This is a double blind, randomized trial examining combination therapy versus single agent therapy with long term (e.g. at least 12 months) follow-up on the last patient randomized. All patients will remain on therapy until the last patient completes the study. All patients will then be transitioned, based on the findings, to open label of combination with continued follow-up or some recommendation about single agent therapy. The primary interest is in combination therapy. Therefore, a two-group combination versus single agent concept will be used—splitting the population into single agent and combination therapy equally. The single agent arm is divided into two groups, ibudilast and cladribine providing for 3 treatment arms: ibudilast and cladribine (50% of the patients), ibudilast and placebo (25% of the patients) and cladribine and placebo (25% of the patients). The general exclusion criteria is followed.

### Example 7

### A Double-Blind, Randomized Study Comparing the Combined Use of Ibudilast and Dimethylfumarate to Either Agent Alone in Patients with Multiple Sclerosis

This is a double blind, randomized trial examining combination therapy versus single agent therapy with long term (e.g. at least 12 months) follow-up on the last patient randomized. All patients will remain on therapy until the last patient completes the study. All patients will then be transitioned, based on the findings, to open label of combination with continued follow-up or some recommendation about single agent therapy. The primary interest is in combination therapy. Therefore, a two-group combination versus single agent concept will be used—splitting the population into single agent and combination therapy equally. The single agent arm is divided into two groups, ibudilast and dimethylfumarate providing for 3 treatment arms: ibudilast and dimethylfumarate (50% of the patients), ibudilast and placebo (25% of the patients) and dimethylfumarate and placebo (25% of the patients). The general exclusion criteria is followed.

### Example 8

### Suppression of IL-5 and/or IL-13 Production with PDE Inhibitors

In order to evaluate candidate agents that protect against glatiramer acetate-induced elevation of inflammatory products, the phosphodiesterase inhibitor, e.g. ibudilast is evaluated by culturing microglia at a concentration of 1×10⁶/ml in 24-well culture plates with 300 μM/mL GA and/or 1 μg/ml LPS (lipopolysaccharide), in the presence of 1 μM/mL-300 μM/mL ibudilast for 24 hours. Supernatants are then collected and assessed for the cytokine contents by ELISA and for NO by Griess method. Further experiments will demonstrate that PDEIs can suppress IL-5 and/or IL-13 production by microglia T cells induced by GA.

From the foregoing it will be appreciated that, although specific embodiments of the invention have been described herein for purposes of illustration, various modifications may be made without deviating from the spirit and scope of the invention claimed.

What is claimed is:

1. A method of treating a patient suffering from the negative effects of multiple sclerosis, said method comprising administering a therapeutically effective amount of at least one phosphodiesterase inhibitor, its pharmacologically acceptable salt, or a hydrate or solvate of one of the foregoing (collectively, “PDE inhibitor”), and a therapeutically effective amount of at least one immunomodulator comprising Mitoxantrone, Natalizumab, Fingolimod, Laquinimod, Cladribine, Dimethylfumarate or a mixture comprising synthetic polypeptide analogs of myelin basic protein, including alanine, glutamic acid, lysine, and tyrosine amino acid residues.

2. The method of claim 1, wherein the administering step comprises simultaneously administering the at least one PDE inhibitor and the at least one immunomodulator.

3. The method of claim 1, wherein the administering step comprises administering the at least one PDE inhibitor and the at least one immunomodulator in close temporal proximity.

4. The method of claim 1, wherein the mixture comprises glatiramer acetate.

5. The method of claim 1, wherein the at least one pharmacologically acceptable salt comprises an inorganic acid addition salt, an organic carboxylic acid addition salt, or a combination thereof.

6. The method of claim 1, wherein the administering step comprises administering the at least one PDE inhibitor and the at least one immunomodulator in a form of a solution or a suspension.

7. The method of claim 6, wherein the solution or suspension further comprises at least one of a sterile diluent, an antibacterial agent, an antioxidant, a chelating agent, a buffer, a tonicity adjusting agent, or combinations thereof.

8. The method of claim 1, wherein the administering step comprises orally administering the at least one PDE inhibitor and the at least one immunomodulator.

9. The method of claim 8, wherein the at least one PDE inhibitor and the at least one immunomodulator are administered in a form of a tablet or a capsule.

10. The method of claim 1, wherein the at least one PDE inhibitor comprises ibudilast.

11. A composition for treating a patient suffering from the negative effects of multiple sclerosis comprising: at least one phosphodiesterase inhibitor, its pharmacologically acceptable salt, or a hydrate or solvate of one of the foregoing (collectively, “PDE inhibitor”), and at least one immunomodulator comprising mitoxantrone, natalizumab, fingolimod, laquinimod, cladribine, dimethylfumarate or a mixture comprising synthetic polypeptide analogs of myelin basic protein, including alanine, glutamic acid, lysine, and tyrosine amino acid residues.

12. The composition of claim 11, wherein the mixture comprises glatiramer acetate.

13. The composition of claim 11, wherein the at least one pharmacologically acceptable salt comprises an inorganic acid addition salt, an organic carboxylic acid addition salt, or a combination thereof.

14. The composition of claim 13, wherein the inorganic acid addition salt is selected from one or more mineral acid addition salts.

15. The composition of claim 14, wherein the one or more mineral acid addition salts is selected from a hydrochloric acid addition salt, a sulfuric acid addition salt and a nitric acid addition salt.

16. The composition of claim 13, wherein the organic carboxylic acid addition salt is selected from one or more of an acetic acid addition salt, a propionic acid addition salt, a maleic acid addition salt, a fumaric acid addition salt, an oxalic acid addition salt, a carboxysuccinic acid addition salt and a citric acid addition salt.

17. The composition of claim 11, which is in a form of a solution or a suspension.

18. The composition of claim 17, wherein said solution or suspension further comprises at least one of a sterile diluent, an antibacterial agent, an antioxidant, a chelating agent, a buffer, a tonicity adjusting agent or combinations thereof.

19. The composition of claim 11, which is in a form of a tablet or a capsule suitable for oral administration.

20. The composition of claim 11, wherein the at least one PDE inhibitor is administered in a solution or suspension at a concentration ranging from about 1 μM/mL to about 300 μM/mL and the mixture comprising synthetic polypeptide analogs of myelin basic protein is administered in the same solution at a specific activity ranging from about 1 mg/mL to about 100 mg/mL.

21. The composition of claim 11, wherein the at least one PDE inhibitor is administered in a tablet or capsule at a dosage ranging from about 20 mg to about 120 mg and the mixture comprising synthetic polypeptide analogs of myelin basic protein is administered in the same tablet or capsule at a specific activity ranging from about 1 mg to about 100 mg.

22. The composition of claim 11, wherein the at least one PDE inhibitor and the mixture comprising synthetic polypeptide analogs of myelin basic protein are administered in a solution in which the foregoing active ingredients are present in a weight ratio ranging from about 1:5 to about 5:1.

23. A method of modulating effects of a mixture comprising synthetic polypeptide analogs of myelin basic protein on microglial production of an inflammatory mediator in a patient, comprising: co-administering at least one PDE inhibitor and a mixture comprising synthetic polypeptide analogs of myelin basic protein in an amount sufficient to reduce an increase in microglial production of an inflammatory mediator induced by the mixture comprising synthetic polypeptide analogs of myelin basic protein.

24. The method of claim 23, wherein the inflammatory mediator comprises Interleukin-5 (IL-5).

25. The method of claim 23, wherein the inflammatory mediator comprises IL-13.

26. The method of claim 23, wherein the microglial production is stimulated by lipopolysaccharide.

27. The method of claim 23, wherein the at least one PDE inhibitor is administered in a solution at a concentration ranging from about 1 μM/mL to about 300 μM/mL and the mixture comprising synthetic polypeptide analogs of myelin basic protein is administered in the same solution at a specific activity ranging from about 1 mg/mL to about 100 mg/mL.

28. The method of claim 23, wherein the at least one PDE inhibitor is administered in a tablet or capsule at a dosage ranging from about 20 mg to about 120 mg and the mixture comprising synthetic polypeptide analogs of myelin basic protein is administered in the same tablet or capsule at a specific activity ranging from about 1 mg to about 100 mg.

29. The method of claim 23, wherein the at least one PDE inhibitor and the mixture comprising synthetic polypeptide analogs of myelin basic protein are administered in a solution in which the foregoing active ingredients are present in a weight ratio ranging from about 1:5 to about 5:1.

30. The method of claim 23, in which the patient is suffering from the negative effects of multiple sclerosis.

31. The method of claim 23, wherein the at least one PDE inhibitor comprises ibudilast.
